(19) United States
(12) Patent Application Publication
(10) Pub. No.: US 2011/0137195 A1
(43) Pub. Date: Jun. 9, 2011

(54) Method and Apparatus for Monitoring Respiratory Activity

(75) Inventors: Esther Rodriquez-Villegas, London (GB); Philip George Corbishley, Oxford (GB); John Sidney Duncan, Bucks (GB); Shalagh Jean Macsorley Smith, Berkshire (GB)

(73) Assignee: IMPERIAL INNOVATIONS LIMITED, London (GB)

(21) Appl. No.: 12/280,835

(22) PCT Filed: Feb. 28, 2007
(86) PCT No.: PCT/GB2007/000701

§ 371 Date: Mar. 16, 2010

(30) Foreign Application Priority Data
Mar. 1, 2006 (GB) 0604138.8

(51) Int. Cl. A61B 5/08

(52) U.S. Cl. 600/529

(57) ABSTRACT

A method of monitoring respiratory activity includes obtaining a respiration sound signal. The respiration sound signal is processed to identify a frequency component corresponding to respiration, and an amplitude related parameter of the component is measured. If the measured parameter is in an indeterminate region then a temporal domain related parameter of the identified frequency component is measured. An apparatus for monitoring respiratory activity is also disclosed.

The invention relates to a method and apparatus for monitoring respiratory activity.

Monitoring respiratory activity is of importance, for example, in detecting apnea, that is, cessation of breathing which can occur during and after an epileptic seizure, in sudden infant death syndrome (SIDS) and in other instances.

Various monitoring schemes have been proposed in the past. For example U.S. Pat. No. 6,666,830 describes the use of a microphone for detecting respiratory sound. The detected sound is compared with samples stored in memory to identify predetermined respiratory patterns. However this arrangement requires both memory storage and complex and processing-heavy signal comparison steps which require a bulky apparatus with high power consumption.

U.S. Pat. No. 4,306,567 discloses an arrangement including a microphone for detecting respiratory sound and a band pass filter which passes that part of the signal in a predetermined band. The arrangement further includes a pulse discriminator to ensure that the filtered signal is of the correct duration. An integrator is used to measure the duration of the signal and, from this, it is determined whether respiration is taking place or not. However, once again, this arrangement requires complex processing steps and hence has high power consumption.

The invention is set out in the claims. According to the invention a respiration sound signal is processed to identify a frequency component corresponding to respiration and the duration of that component is measured if its amplitude or a related parameter is in an indeterminate region. As a result, the additional processing is only required if the signal is within a certain amplitude band reducing the processing components and hence power consumption.

Embodiments of the invention will now be described with reference to the drawings of which:

FIG. 1 is a schematic diagram showing use of a respiration monitoring apparatus;

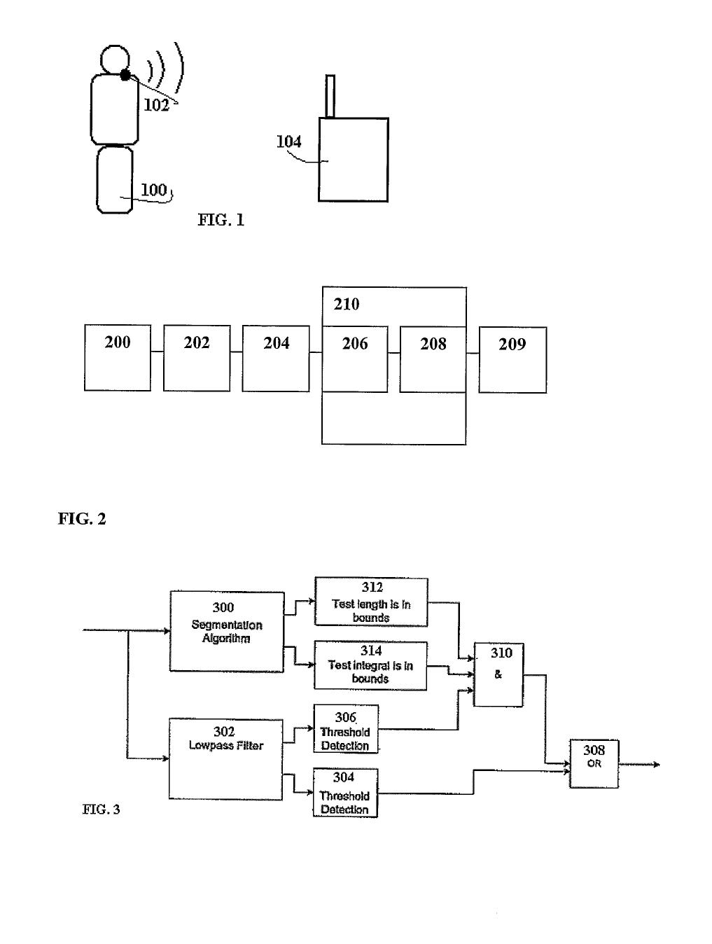

FIG. 2 is a block diagram showing components of a respiration monitoring apparatus;

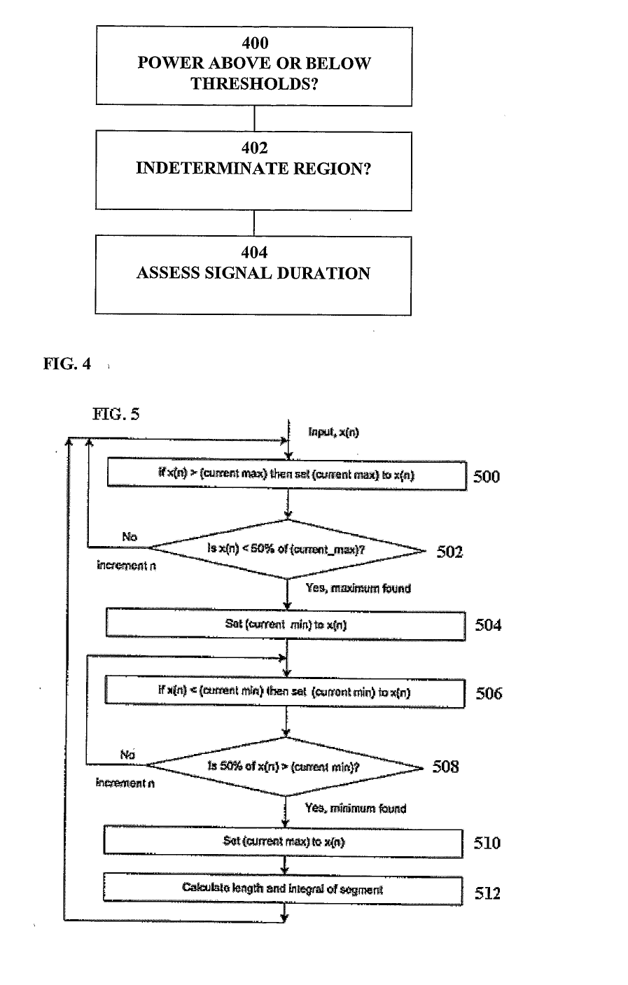

FIG. 3 is a block diagram showing in more detail one aspect of a respiration monitoring apparatus;

FIG. 4 is a flow diagram showing steps involved in monitoring respiratory activity;

FIG. 5 is a flow diagram showing the additional steps involved in monitoring respiratory activity; and

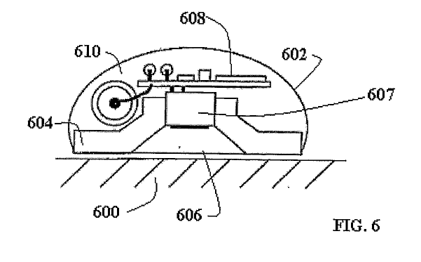

FIG. 6 is a schematic diagram showing the structure of a respiration monitoring apparatus.

Referring firstly to FIG. 1 an individual 100 whose respiratory activity is monitored carries a respiration monitor 102 arranged to monitor respiratory activity and, for example, detect apnoea. The monitor 102 sends a signal wirelessly representing respiratory activity to a base station 104 which then takes appropriate action as required. For example, where the monitor 102 detects cessation of breathing an alarm signal is raised at the base station 104 for example, by sending an alarm to a hospital or a home alarm system.

The monitor 102 is designed to be compact, lightweight and to have a long battery lifetime such that it does not interfere with the activity of the patient 100

In an alternative configuration the monitor 102 comprises means for providing a human detectable signal to the wearer 100.

The principal components of the monitor 102 can be seen in FIG. 2. A respiration sound signal is received at a detector 200 comprising a transducer such as a microphone. The detector sends a signal to a band pass filter 202 which passes frequencies in the range which is found to be a representative frequency band for respiratory sounds, hence allowing identification of frequency components of the detected signal corresponding to respiration. The filtered signal is passed to a rectifier 204 for example comprising a full-wave diode rectifier and the rectified signal is smoothed at low pass filter 206. The smoothed and rectified signal passes through a feature recognition section 210 including a comparator 208 to detect whether an amplitude related parameter of the filtered signal such as signal power meets a predetermined threshold and if so a signal is passed to a transmitter 209 which sends an appropriate signal. For example a signal can be sent continuously if respiration is detected, stopping if respiration ceases, or an alarm signal can be sent in the case of cessation of respiration or the device becoming dislodged. The transmitter can be, for example, arranged to transmit an electrical stimulus, electrical pulse, electromagnetic wave, infrared signal and the alarm or any other form of transmission which may be received by the human body or a receiver.

In operation therefore, the input signal is amplified and band pass filtered, to provide the frequency component corresponding to respiration and then low pass filtered. The processed signal is then passed through the comparator to determine whether it is above or below a given threshold. In particular it is necessary to establish whether the signal is of large enough magnitude to represent breathing. The threshold itself can be determined empirically or will be apparent to the skilled reader for example using a calibration phase and if the signal is below the threshold then breathing is not considered to be taking place.

In this case a signal is sent to the transmitter block, from the comparator if the signal is above the threshold and the transmitter block then sends a signal indicating that the received signal is above the threshold. For example the transmitter block then sends a signal once every second indicating that the respiration signal is above the threshold. In this case cessation of the transmitted signal for a predetermined period is detected by the receiving base station as an indication that breathing has stopped, allowing appropriate alarm signals to be sent out. An advantage of this approach is that because the alarm system relies on the absence of an otherwise present signal, failure of the respiration monitor or power loss will also be detected which would not be the case if the base station were expecting an alarm signal only upon cessation of respiration from the transmitter. In an alternative approach, if breathing is not detected an appropriate signal is sent to the transmitter circuit to send an alarm signal. Similarly, if the signal is above the threshold then breathing is considered to be taking place and the transmitter circuit is not activated.

In practice the device is positioned on the body of the patient and may be attached in any way, for example, using glue, gel, strapping or tape. In one preferred approach glue is used to avoid noise artefacts associated with the use of tape, for example, arising from body or air movement. The microphone includes a hollow air chamber which is sealed to the skin and which acoustically couples bio-acoustic sounds from the body to the microphone which are then processed, as described above. Alternatively, the acoustic chamber can be enclosed with a membrane, the membrane then making contact with the patient. The entire device can be coated in a foam plastic or other found damping material to attenuate acoustic signals from the environment.

Referring to FIG. 6, for example, the device is attached to a surface such as the skin of the patient 600 and comprises a housing 602, one or more microphones 607 defining an acoustic chamber 606 and, mounted thereon, electronic circuitry of the type described above, for example, on an appropriate circuit board 608 including a transmitter 610 for transmitting to a base station (not shown). The device is powered by a battery of any appropriate kind (not shown).

The location of the microphone is found to be extremely important as a result of the filtering effects of human tissue and the relative location of the sound sources. In particular the microphone is most preferably located at the neck and in particular at the suprasternal notch of the neck which is found to provide a particularly improved signal.

As discussed above, filtering steps are introduced to reject noise and artefacts in the respiratory sound signal and, if the average power or other amplitude related parameter is below a minimum value there can be assumed to be no breathing whereas if it is above a maximum it can only be caused by air flow or speech in the user such that breathing may be assumed.

However in an optimisation, the approach described herein further analyses an indeterminate region where the signal power is neither large enough to definitely be a breathing signal nor small enough to be assumed to be noise. The approach can be understood generally with reference to FIG. 3 which is a block diagram showing the feature recognition aspects 210 of FIG. 2 in more detail, in conjunction with the flow diagram of FIG. 4. At step 400, if the power is either above or below the respective thresholds processing proceeds as described above. However, if at step 402 the power is in the band between those thresholds i.e. an indeterminate region, then at step 404 the duration of the signal is assessed.

In particular, referring to FIG. 3, the filtered and rectified respiration sound signal is passed to each of a segmentation algorithm 300 and a first order low pass filter. The low pass filtered signal from block 302 passes to each of an upper and lower threshold detection component 304, 306 respectively. Where the signal power is above the upper threshold then threshold detector 304 passes the signal to an OR gate 308 from which it is passed to the transmitter as described in more detail above. If the average power is below the lower threshold then threshold detector 306 passes a NULL output to an AND gate 310. The other inputs to the AND gate are received from the segmentation algorithm such that, in this case the segmentation algorithm is switched off

However, in case that the signal power is in the indeterminate region between the upper and lower thresholds, then the segmentation algorithm 300 is implemented to allow distinction between artefacts and a breathing signal Because the artefacts tend to be transient signals, with similar harmonic characteristics but different temporal characteristics, the segmentation algorithm applies a time domain technique. In particular, taking into account that breaths comprise an inhalation and exhalation phase, each of which have bounds in terms of their minimum and maximum lengths, integrals and other parameters, the segmentation distinguishes between transients and a breathing signal accordingly. As a result, breaths can be discriminated from artefacts and noise which tend to be of considerable power but short in length, or are of less power and are longer in length.

In particular at blocks 312 and 314 it is tested whether the signal length and signal integral are within certain bounds as can be understood in more detail with reference to the flow diagram of FIG. 5. As a first step it is necessary to find local minima in order to provide the start and end points of the part of the signal to be assessed. In the first subroutine, a maximum is identified by testing values sample by sample. At step 500, upon receipt of an input sample signal power value x(n), x(n) is compared against a value (current max). If x(n) exceeds (current max) then the value of (current max) is set to the sampled value of x(nₘₐₓ). At step 502 x(n) is tested to see whether it is less than 50% off (current max). If it is not then n is incremented, and the next sample x(n+1) is tested once again. However, if, at step 502, x(n) is less than 50% of (current max) then this indicates that the sampled power has dropped to less than half of the maximum value (current max) such that (current max) for the respective sample x(nₘₐₓ) is identified.

Having identified the maximum value and position the algorithm seeks a minimum. In particular the value x(n) passed through step 502 is used to set the value (current min) at step 504. At step 506 a sub routine is commenced such that where x(n) is less than (current min) then the value of (current min) is set to x(nₘᵢₙ). Then, at step 508, if 50% of the value of x(n) is greater than (current min) that is to say, (current min) is half of x(n) then a minimum is considered to be found at x(nₘᵢₙ), i.e. x(n) has increased from the minimum value. If not, however, n is incremented and step 506 is repeated for x(n+1).

In the case where a minimum has been found then at step 510 the maximum seeking sub routine is reinitiated by setting (current max) to the value of x(nₘᵢₙ) from step 508.

At step 512, the minima found are used to divide the signal into segments from which parameters of the segments may be extracted, that is to say, those parts of the signal bounded by minima are considered to be useful parts of the signal. Those parts can be assessed in any particular way, for example, using integral length, central gravity and harmonics. In one optimisation the two parameters adopted are the integral of the signal and the length which are advantageously found to be the most orthogonal or independent of one another and are also simple to calculate and implement in analogue circuits. If the integral and length of a segment are within bounds it is considered to be a breath and a signal is sent to the AND gate 310. As a result the AND gate passes a signal if both the test length and test integral from blocks 312 and 314 are within bounds. The signal is sent to the OR gate 308 and then to the output.

Another approach which improves the performance of the device in noisy environments involves a second microphone outside the acoustic chamber. Using adaptive signal processing, for example signal subtraction, the signal from this microphone is used to remove ambient noise from the chamber centre which picks up the patient signal.

It will be recognised by the skilled reader that the components used in the arrangement described above may be of any appropriate form. For example the circuitry may be in the form of a digital signal processing (DSP) block, a microprocessor, custom logic, or a field processor gate array (FPGA). Advantageously, in order to minimise power consumption, the circuitry may be implemented in analogue electronics.

For example the band pass filter may be a second order elliptic filter—a low order filter is advantageous as filter power consumption is proportioned to the number of poles or order. Also, ripples found in the pass band of an elliptic filter are not critically important in view of the signal processing according to the method described herein.

Similarly rectification may be implemented using any appropriate system, for example a Hilbert rectifier or a full wave diode rectifier. Diode rectification is easily implemented in analogue circuitry and low pass filtering of the signal can provide similar advantages to those of a Hilbert rectifier. For the signal smoothing step, removing harmonics due to the carrier frequency and rectification process, a low pass filter of any appropriate type can be adopted with a heuristically selected cut off to provide a balance between a loss of higher order harmonics in the signal and a degradation of signal to noise ratio due to high frequency artefacts.

The transmission scheme used by the transmitter circuit may be any appropriate scheme for example on-off keying (OOK) where the transmitted signal is formed from 16 consecutive values allowing identification of the specific device emitting the signal.

As a result of the arrangement described herein the device can be compact and have a long battery life as a result of low power consumption.

It will be appreciated that in addition the device may contain additional parts such as ECG monitoring systems, systems to process the microphone signal in alternative ways, systems for transmission and systems to provide stimuli to the body. It will be further appreciated that the approaches described herein can be applied to monitoring respiratory activity for other purposes than detecting apnoea, such as tachypnoea, or irregularities of breathing rhythm. Yet further the approaches described herein can be applied to monitoring of other biological activity where a signal having both recognisable frequency domain and time domain components are derivable, for example, cardiac activity, electrical activity emanating from the central nervous system, or electrical activity deriving from muscle or peripheral nerve.

1. A method of monitoring respiratory activity comprising obtaining a respiration sound signal, processing the respiration sound signal to identify a frequency component corresponding to respiration, measuring an amplitude related parameter of the identified frequency components and, if the parameter is in an indeterminate region, measuring a temporal domain related parameter of the identified frequency component.

2. A method as claimed in claim 1 in which, if the amplitude related parameter falls below a lower limit of the indeterminate region then a cessation of respiration state is identified and if the amplitude related parameter exceeds an upper limit of the indeterminate region, then a respiration continuation state is identified.

3. A method as claimed in claim 1 in which the temporal domain related parameter comprises at least one of duration and integral of the identified frequency component.

4. A method as claimed in claim 1 in which, if the temporal domain related parameter is measured to be within a respiration range then a respiration continuation state is identified and if the parameter is outside the respiration range then a cessation of respiration state is identified.

5. A method as claimed in claim 3 in which, if cessation of breathing is detected then an apnoea alarm is triggered.

6. A method as claimed in claim 1 in which the amplitude related parameter comprises signal power.

7. A method as claimed in claim 1 in which the respiration sound signal is processed to identify a frequency component comprising a frequency pass band.

8. A method as claimed in claim 7 in which the frequency pass band is in the region of approximately 500 to 900 hertz.

9. A method as claimed in claim 1 in which the temporal domain related parameter is measured for a signal segment.

10. A method as claimed in claim 9 in which the signal segment is defined between adjacent maxima or adjacent minima.

11. A method as claimed in claim 10 in which the adjacent maxima or adjacent minima are detected by a peak detection algorithm.

12. A method as claimed in claim 1 in which ambient noise is detected and filtered out of the respiration sound signal.

13. A method as claimed in claim 12 in which the ambient noise is filtered out by signal subtraction.

14. An apparatus for monitoring respiratory activity comprising a respiration sound monitor and a processor arranged to identify a frequency component corresponding to respiration from the respiration sound signal, measure an amplitude related parameter of the identified frequency component and, if the parameter is in an indeterminate region, measure a temporal-domain related parameter of the identified frequency component.

15. An apparatus as claimed in claim 14 in which the monitor comprises at least one microphone.

16. An apparatus as claimed in claim 14 in which the processor includes a band pass filter for identifying the frequency components corresponding to respiration.

17. An apparatus as claimed in claim 14 in which the processor further comprises a transmitter for transmitting a monitoring signal wirelessly.

18. An apparatus as claimed in claim 14 in which the apparatus is configured to be located at the supra sternal notch of a patient's neck, and also over the trachea, and elsewhere on the neck or body, as may best suit individual subjects.

19. An apparatus as claimed in claim 14 in which the apparatus is configured to be glued taped, gel-adhered or strapped to a monitoring surface.

20. An apparatus as claimed in claim 14 in which the respiration sound monitor includes an acoustic chamber having an end arranged to be placed against a respiration sound source.

21. An apparatus as claimed in claim 20 in which the acoustic chamber end is open.

22. An apparatus as claimed in claim 20 in which the acoustic chamber end is closed by a member.

23. An apparatus as claimed in claim 14 in further comprising an ambient noise monitor.

24. An apparatus as claimed in claim 23 when dependent on claim 20 in which the ambient noise monitor is located external to the acoustic chamber.

25. (canceled)
